(19) United States
(12) Patent Application Publication
(10) Pub. No.: US 2010/0226999 A1
(43) Pub. Date: Sep. 9, 2010

(54) PROCESS FOR FORMING STABILIZED OPHTHALMIC SOLUTIONS

(76) Inventor: Tracy Quevillon-Coleman, Jacksonville, FL (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/399,647

(22) Filed: Mar. 6, 2009

(51) Int. Cl. A61K 33/40; A61K 47/12; A61K 47/02; A61K 31/4535

(52) U.S. Cl. 424/616; 514/784; 514/770; 514/324

(57) ABSTRACT

The present invention relates to a method for forming an ophthalmic composition comprising mixing at least one hydroxide and pentetic acid with water and at least one oxidatively unstable component to form an ophthalmic composition comprising at least one salt of diethylenetriamine pentaacetic acid and a pH between about 6 and about 8.

### BACKGROUND OF THE INVENTION

There are many commercially available ophthalmic solutions. The solutions should provide disinfection against a variety of bacteria and fungi, which can come in contact with the eye and devices which reside on the eye, such as contact lenses. The solutions must remain free from contamination during the use life of the solution. To meet this requirement solutions either contain a preservative component or are sterile packaged in single use dosages. For contact lens cleaning and care solutions, and over the counter eye drops, multidose containers are popular. These solutions require the inclusion of preservatives (for eye drops) and disinfecting compositions (for contact lens cleaning and care solutions).

Salts of diethylenetriamine pentaacetic acid (DTPA) comprising at least one calcium salt, zinc salt or mixed calcium/zinc salt of diethylenetriamine pentaacetic acid have been disclosed to be useful as stabilizers for a variety of ophthalmic solutions. The DTPA salts are manufactured by reacting pentetic acid (diethylenetriaminepentaacetic acid) with two equivalents of the desired alkali metal hydroxide in water. The product is then isolated from water by azeotropic distillation with heptane and dried under vacuum to give a white to yellow powder. However, Ca₂DTPA is extremely hygroscopic and difficult to dry, handle and store as a dry powder. Water impurity in Ca₂DTPA negatively impacts the efficacy of the Ca₂DTPA. Heptane is a class III solvent and is always present at residual levels in Ca₂DTPA.

Thus, it would be desirable to eliminate the handling issues with Ca₂DTPA and to have a process which did not use heptane.

### SUMMARY OF THE INVENTION

The present invention relates to a process for forming ophthalmic solutions comprising at least one salt of DTPA.

The present invention further relates to a process comprising mixing at least one alkali metal hydroxide and pentetic acid with water and at least one oxidatively unstable component.

### DESCRIPTION OF THE INVENTION

The present invention relates to process for forming ophthalmic solutions comprising at least one salt of DTPA.

As used herein storage stable, means that under storage conditions, such as temperatures of less than about 40° C., the solution loses less than thirty percent of its efficacy over thirty days, and in some embodiments less than about 25% in thirty days.

The term “ophthalmic composition” refers to liquids, aerosols, or gels that may be topically administered to the eye.

The term “stabilizing agent” refers to chelant compositions that inhibit metal catalyzed oxidative degradation of the oxidatively unstable pharmaceutical ingredient. Examples of stabilizing agents include but are not limited to silica, chitin derivative such as chitosan, polyamides such as poly(aspartic acid-co-ω-amino acid (See CAN:129:54671, Shibata, Minako et al. Graduate School Environmental Earth Science, Hokkaido University, Sapporo, Japan Macromolecular Symposia (1998), 130, 229-244) and polymeric amides such as poly[iminocarbonyl(2,5-dihydroxy-1,4-phenylene)carbonylimino-1,4-phenylenemethylene-1,4-phenylene], CAS #87912-00-3, polymeric lactams such as polyvinylpyrrolidone, polyamino carboxylic acids such as diethylenetriaminepentaacetic acid and triethylenetriaminepentaacetic acid, polymeric amines such as polyallylamine, crown ethers such as 18-crown-6,21-crown-7, and 24-crown-8, cellulose and its derivatives, and N,N,N′,N′,N″,N″-hexa(2-pyridyl)-1,3,5-tris(aminomethyl)benzene, and certain macrocyclic ligands such as crown ethers, ligand containing knots and catenands (See, David A. Leigh et al Angew. Chem. Int. Ed., 2001, 40, No. 8, pgs. 1538-1542 and Jean-Claude Chambron et al. Pure & Appl. Chem., 1990, Vol. 62, No. 6, pgs. 1027-1034). At least one of the stabilizing agents in the present invention is a salt of DTPA such as CaNa₃DTPA, ZnNa₃DTPA, and Ca₂DTPA.

The term “effective amount” refers to the amount of stabilizing agent required to inhibit the oxidative degradation of the oxidatively unstable component. In most circumstances it is preferred that there is a 1:1 molar ration of metal present in the ophthalmic composition to chelant, is more preferably about 1 of metal to greater than about 1 of chelant compositions, most preferably about 1 of metal to greater than or equal to about 2 of chelant compositions.

Ophthalmic compositions are any composition which can be directly instilled into an eye, or which can be used to soak, clean, rinse, store or treat any ophthalmic device which can be used placed in or on the eye. Examples of ophthalmic compositions that may be topically administered to the eye, ophthalmic device packing solutions, cleaning solutions, conditioning solutions, storage solutions, eye drops, eye washes, as well as ophthalmic suspensions, aerosols, gels and ointments and the like. In one embodiment of the present invention, the ophthalmic composition is an ophthalmic solution.

Ophthalmic devices include any devices which can be placed on the eye, or any part of the eye, such as, but not limited to under the eyelid or in the punctum. Examples of ophthalmic devices include contact lenses, ophthalmic bandages, ophthalmic inserts, punctal plugs and the like.

The ophthalmic compositions of the present invention are formed by mixing at least hydroxide and pentetic acid with the desired carrier and other ophthalmic composition components.

Suitable hydroxides include ophthalmically compatible cations, such as sodium, calcium, zinc, magnesium and mixtures thereof. In one embodiment, the ophthalmically compatible cation comprises calcium. Examples of hydroxides include calcium hydroxide, sodium hydroxide, zinc hydroxide, magnesium hydroxide, mixtures thereof and the like. In one embodiment, the hydroxides of the present invention are substantially free from oxidating transition metals such as iron, nickel, copper and manganese. As used herein, substantially free means a concentration less than the chelating capacity of the penetrate salt, such that oxidative degradation of any oxidatively unstable components included in the ophthalmic compositions of the present application is avoided.

The hydroxide is added in amounts suitable to generate between about 0.005 wt % (50 ppm) to about 0.15 wt % (1500 ppm), and in some embodiments from about 100 to about 1000 ppm, and in yet another embodiment between about 50 and about 500 ppm of the desired salt of diethylenetriamine pentaacetic acid. In one embodiment suitable amounts of pentetic acid include from about 40 ppm to about 825 ppm and from about 15 ppm to about 310 ppm hydroxide. For example, for a solution where 100 ppm Ca₂DTPA was desired, about 0.0084 wt % Ca(OH)₂ and 0.0032% pentetic acid are added to the desired carrier and other ophthalmic composition components.

The ophthalmic compositions are mixed until all components have been incorporated. For ophthalmic solutions, the components are mixed until all components are dissolved. Suitable mixing times may vary depending upon the specific components selected, but can range from about 1 minute to about 2 hours and in some embodiments from about 15 minutes to about 1 hour. Mixing may be conducted at ambient or elevated temperature and is conveniently done from about 20 to about 40° C.

The ophthalmic compositions may be further processed and packaged using known methods, including, but not limited to filtration, sterilizing filtration and the like. Ophthalmic compositions which are heat stable may be sterilized by autoclaving, while thermally instable ophthalmic compositions may be sterilized via irradiation, or may be aseptically packaged.

Dicalcium diethylenetriamine pentaacetic acid has been found to be at least as effective, and at some concentrations more effective at stabilizing compositions comprising at least one oxidatively unstable component than diethylenetriamine pentamethylenephosphonic acid (DTPPA) or EDTA. Dicalcium diethylenetriamine pentaacetic acid is also less cytotoxic and has a more neutral pH than does DTPPA.

The ophthalmic compositions of the present invention also have a pH of between about 6 and 8, and in some embodiments between about 6.5 and about 7.5. This allows the compositions of the present invention to be instilled directly in the eye, and to be used on ophthalmic devices that are to be placed in the ocular environment.

The ophthalmic compositions of the present invention further comprise at least one oxidatively unstable component. Oxidatively unstable components are unstable in the presence of oxygen and certain transition metals. Oxidatively unstable components include hydrogen peroxide, oxidatively unstable excipients, oxidatively unstable pharmaceutical compounds used to treat conditions of the eye, and combinations thereof.

As used herein “oxidatively unstable excipient” refers to a component of ophthalmic compositions that degrades in the presence of oxygen and certain transition metals. Examples of unstable excipients include but are not limited to astringents, demulcents, emollients, hypertonicity agents, oleaginous, agents, tonicity agents mucomimetic agents, and the like. Particularly examples of unstable excipients include but are not limited to cellulose derivatives, carboxymethylcellulose, hydroxyethylcellulose, hydroxypropylcellulose, hyaluronic acid, methylcellulose, Dextran, gelatin, polyols, glycerin, polyethylene glycol, polysorbate, propylene glycol, polyvinyl alcohol, povidone lanolin, mineral oil, paraffin, petrolatum, white ointment, white petrolatum, white, wax, and yellow wax.

Examples of pharmaceutical compounds include antihistamines, antibiotics, antibacterial agents, antiviral agents, antifungal agents, analgesics, anesthetics, antiallergenic agents, mast cell stabilizers, steroidal and non-steroidal anti-inflammatory agents, angiogenesis inhibitors; antimetabolites, fibrinolytics, neuroprotective drugs, angiostatic steroids, mydriatics, cyclopegic mydriatics; miotics; vasoconstrictors; vasodilators, anticlotting agents; anticancer agents, antisense agents, immunomodulatory agents, carbonic anhydrase inhibitors, integrin antabonistsl; cyclooxygenase inhibitors, VEGF antagonists; immunosuppressant agents and the like. Particularly, examples of pharmaceutical agents include but are not limited to acrivastine, antazoline, astemizole, azatadine, azelastine, buclizine, bupivacaine, cetirizine, clemastine, cyclizine, cyproheptadine, ebastine, emedastine, ephedrine, eucatropine, fexofenadine, homatropine, hydroxyzine, ketotifen, levocabastine, levoceterizine, lomefloxacin, meclizine, mepivacaine, mequitazine, methdilazine, methapyrilene, mianserin, naphazoline norastemizole, norebastine, ofloxacin, oxymetazoline, pheniramine, phenylephrine, physostigmine, picumast, promethazine, scopolamine, terfenadine, tetrahydrozoline, thiethylperazine, timolol, trimeprazine, triprolidine, pharmaceutically acceptable salts and mixtures thereof. In one embodiment the oxidatively unstable pharmaceutical compounds include acrivatine, antazoline, astemizole, azatadine, azelastine, clemastine, cyproheptadine, ebastine, emedastine, eucatropine, fexofenadine, homatropine, hydroxyzine, ketotife, levocabastine, levoceterizine, meclizine, mequitazine, methdialazine, methapyrilene, norastemizole, norebastine, oxymetazoline, physootigmine, picumast, promethazine, scopolamine, terfenadine, tetrahydrozoline, fimilol, trimeprazine, triprolidine, and pharmaceutically acceptable salts thereof. In another embodiment the oxidatively unstable pharmaceutical compounds include phenarimine, ketotifen, ketotifen fumarate nor ketotifen, olapatadine and mixtures thereof. In yet another embodiment the oxidatively unstable compound comprises ketotifen, its pharmaceutically acceptable salts, and mixtures thereof.

Examples of nutraceutical compounds include vitamins and supplements such as vitamins A, D, E, lutein, zeaxanthin, lipoic acid, flavonoids, ophthalmically compatible fatty acids, such as omega 3 and omega 6 fatty acids, combinations thereof, combinations with pharmaceutical compounds and the like. Oxidatively unstable pharmaceutical or nutraceutical compounds which benefit most from the present invention are those that degrade when solutions of these compounds and oxidative catalysts (such as metals and metallic salts) are mixed together at ambient or elevated temperatures, as compared to solutions of these compounds without oxidative catalysts at ambient or elevated temperatures. In one embodiment the oxidatively unstable pharmaceutical or nutraceutical compounds degrade at least about 10% when heated to about 120° C. for about 20 minutes with oxidative catalysts. The concentration of oxidatively unstable pharmaceutical ingredients in the ophthalmic compositions of the invention range from about 2 μg/mL to about 0.5 g/mL, particularly preferred, about 0.1 μg/mL to about 10,000 μg/mL.

In another embodiment the ophthalmic composition comprises a multipurpose solution, or contact lens cleaning solution In another embodiment, the ophthalmic composition of the present invention is a multipurpose solution or contact lens cleaning solution comprising between about 50 to about 1000 ppm hydrogen peroxide. In some embodiments the hydrogen peroxide is present in concentrations between about 100 and about 500 ppm, and in other embodiments, between about 100 and about 300 ppm.

Alternatively, the composition may include a source of hydrogen peroxide. Suitable hydrogen peroxide sources are known, and include peroxy compounds which are hydrolyzed in water. Examples of hydrogen peroxide sources include alkali metal perborates or percarbonates such as sodium perborate and sodium percarbonate.

Additional known peroxide stabilizer may also be included, so long as it is not cytotoxic at the concentrations being used, and is compatible with the other ophthalmic composition components. For example, the additional peroxide stabilizer should not interfere with the functioning of any other components included in the composition, and should not react with any other components. Examples of suitable additional peroxide stabilizers include phosphonates, phosphates, ethylene diamine tetraacetic acid, nitrilo triacetic acid, ophthalmically compatible water soluble salts of any of the foregoing, mixtures thereof, and the like. In one embodiment the additional peroxide stabilizer comprises DTPPA or least one pharmaceutically acceptable salt of DTPPA.

The at least one additional peroxide stabilizer may be present in concentrations up to about 1000 ppm, and in some embodiments between about 100 and about 500 ppm. When the additional peroxide stabilizer comprises DTPPA or at least one pharmaceutically acceptable salt of DTPPA, it is present in a concentration up to about 1000 ppm, and in some embodiments between about 100 ppm to about 500 ppm.

The ophthalmic compositions of the present invention may further comprise additional components such as, but not limited to pH adjusting agents, tonicity adjusting agents, buffering agents, active agents, lubricating agents, disinfecting agents, viscosity adjusting agents, surfactants and mixtures thereof. When the ophthalmic composition is an ophthalmic solution, all components in the ophthalmic solution of the present invention should be water soluble. As used herein, water soluble means that the components, either alone or in combination with other components, do not form precipitates or gel particles visible to the human eye at the concentrations selected and across the temperatures and pH regimes common for manufacturing, sterilizing and storing the ophthalmic composition.

The pH of the ophthalmic composition may be adjusted using acids and bases, such as mineral acids, such as, but not limited to hydrochloric acid and bases such as sodium hydroxide.

The tonicity of the ophthalmic composition may be adjusted by including tonicity adjusting agents. In some embodiments it is desirable for the ophthalmic composition to be isotonic, or near isotonic with respect to normal, human tears. Suitable tonicity adjusting agents are known in the art and include alkali metal halides, phosphates, hydrogen phosphate and borates. Specific examples of tonicity adjusting agents include sodium chloride, potassium chloride, calcium chloride, magnesium chloride, zinc chloride, combinations thereof and the like.

The ophthalmic composition may further comprise at least one buffering agent which is compatible with diethylenetriamine pentaacetic acid salt. Examples of suitable buffering agents include borate buffers, phosphate buffers, sulfate buffers, combinations thereof and the like. In one embodiment the buffering agent comprises borate buffer. In another embodiment, the buffering agent comprises phosphate buffer. Specific examples include borate buffered saline and phosphate buffered saline.

The ophthalmic composition may also comprise at least one disinfecting agent. The disinfecting agent should not cause stinging or damage to the eye at use concentrations and should be inert with respect to the other composition components. Suitable disinfecting components include hydrogen peroxide, polymeric biguanides, polymeric quarternary ammonium compounds, chlorites, bisbiguanides, quartemary ammonium compounds and mixtures thereof.

In one embodiment, the ophthalmic composition is an ophthalmic solution comprising hydrogen peroxide and at least one chlorite compound. Suitable chlorite compounds include water soluble alkali metal chlorites, water soluble alkaline metal chlorites and mixtures thereof. Specific examples of chlorite compounds include potassium chlorite, sodium chlorite, calcium chlorite, magnesium chlorite and mixtures thereof. In one embodiment the chlorite compound comprises sodium chlorite.

Suitable concentrations for the chlorite compound include concentrations between about 100 and about 2000 ppm, in some embodiments between about 100 and about 1000 ppm, in other embodiments between about 100 and about 500 ppm and in other embodiments between about 200 and about 500 ppm.

Combinations of suitable peroxide/chlorite disinfecting agents are disclosed in U.S. Pat. No. 6,488,965, U.S. Pat. No. 6,592,907, US20060127497, US2004/0037891, US 2007/0104798. These patents as well as all other patent disclosed herein are hereby incorporated by reference in their entirety.

The ophthalmic compositions of the present invention may further comprise at least one additional disinfecting compound selected from the group consisting of fully saturated, polymeric quaternium salts such as poly[oxyethylene(-dimethylimino)ethylene-(dimethylimino)ethylene dichloride (CAS designation of 31512-74-0, and referred to herein as “Polyquaternium-42”), disclosed in U.S. Pat. No. 5,300,296 and U.S. Pat. No. 5,380,303. The polymeric quaternium salts are desirably fully saturated to insure they are stable in the presence of the hydrogen peroxide. The fully saturated, polymeric quaternium salts may be present in the solution in amounts between about 25 to about 100 ppm. It has been found that when at least one fully saturated, polymeric quaternium salts such as Polyquaternium-42 is included in an ophthalmic solution along with hydrogen peroxide and chlorite the resulting solutions display surprisingly improved antifungal properties, particularly against fusarium solani.

One or more lubricating agents may also be included in the ophthalmic composition. Lubricating agents include water soluble cellulosic compounds, hyaluronic acid, and hyaluronic acid derivatives, chitosan, water soluble organic polymers, including water soluble polyurethanes, polyethylene glycols, combinations thereof and the like. Specific examples of suitable lubricating agents include polyvinyl pyrrolidone (“PVP”), hydroxypropyl methyl cellulose, carboxymethyl cellulose, glycerol, propylene glycol, 1,3-propanediol, polyethylene glycols, mixtures there of and the like. Generally lubricating agents have molecular weights in excess of 100,000. When glycerol, propylene glycol and 1,3-propanediol are used as lubricating agents, they may have molecular weights lower than 100,000.

When a lubricating agent is used, it may be included in amounts up to about 5 weight %, and in some embodiments between about 100 ppm and about 2 weight %.

One or more active agent may also be incorporated into the ophthalmic solution. A wide variety of therapeutic agents may be used, so long as the selected active agent is inert in the presence of peroxides. Suitable therapeutic agents include those that treat or target any part of the ocular environment, including the anterior and posterior sections of the eye and include pharmaceutical agents, vitamins, nutraceuticals combinations thereof and the like. Suitable classes of active agents include antihistamines, antibiotics, glaucoma medication, carbonic anhydrase inhibitors, anti-viral agents, anti-inflammatory agents, non-steroid anti-inflammatory drugs, antifungal drugs, anesthetic agents, miotics, mydriatics, immunosuppressive agents, antiparasitic drugs, anti-protozoal drugs, combinations thereof and the like. When active agents are included, they are included in an amount sufficient to product the desired therapeutic result (a “therapeutically effective amount”).

In embodiments where the ophthalmic compositions of the present invention are cleaning or multipurpose solutions they may also include one or more surfactant, detergent, or mixtures thereof. Suitable examples include tyloxapol, poloxomer (poly(ethylene oxide)-b-poly(propylene oxide)-b-poly(ethylene oxide)) type surfactants which are commercially available from BASF and poloxamine type surfactants (non-ionic, tetrafunctional block copolymers based on ethylene oxide/propylene oxide, terminating in primary hydroxyl groups, commercially available from BASF, under the tradename Tetronic). A specific example is Pluronic F-147 and Tetronic 1304. Tyloxapol is a non-ionic, low molecular weight surfactant, and is fully soluble in the phosphate buffers. Tyloxapol is a detergent commercially available from Pressure Chemical Company. In embodiments where tyloxapol is included, it is included in amounts between about 500 to about 2000 ppm. Surfactants may be used in amounts up to about 5 weight %, and in some embodiments up to about 2 weight %.

Some surfactants may also act as disinfectant enhancers. Disinfectant enhancers for the solutions of the present application include C₅₋₂₀ polyols, such as 1,2-octanediol (caprylyl glycol), glycerol monocaprylate, sorbitan monolaurate (TWEEN 80) combinations thereof and the like. Disinfectant enhancers may be present in amounts from about 50 to about 2000 ppm.

Additionally, the ophthalmic composition may comprise one or more viscosity adjusting agent or thickener. Suitable viscosity adjusting agents are known in the art and include polyvinyl alcohol, polyethylene glycols, guar gum, combinations thereof and the like. The viscosity adjusting agent may be used in amounts necessary to achieve the desired viscosity.

It will be appreciated that all the components at the concentrations they are used herein, will be soluble in buffered solutions, compatible with the other solution components and will not cause ocular pain or damage.

Ophthalmic solutions of the present invention may be formed by mixing the selected components with water. Other ophthalmic compositions may be formed by mixing the selected components with a suitable carrier.

In order to illustrate the invention the following examples are included. These examples do not limit the invention. They are meant only to suggest a method of practicing the invention. Those knowledgeable in contact lenses as well as other specialties may find other methods of practicing the invention. However, those methods are deemed to be within the scope of this invention.

### EXAMPLES

### Comparative Example 1 & Example 1

The ophthalmic solutions shown in Table 1, below were made as follows. The calcium hydroxide and pentetic acid were mixed in the water, and the remaining ingredients listed in Table 1, below were mixed until all of the material dissolved. Six glass vials were filled with 3 mL of each solution. The vials were stopped with poly(tetrafluoroethylene) (“PTFE”) and three of the vials were heated for eighteen minutes at 124° C. Samples of each vial (1.0-1.5 mL) were analyzed by HPLC. The results show that the solutions made by mixing pentetic acid and calcium hydroxide retained 98% of the ketotifen fumarate through 5 sterilization cycles.

TABLE 1

| | Comp. Ex. 1 | Ex. 1 |
|---|---|---|
| Component | (wt %) | (wt %) |
| Water | 98.1457 | 98.1340 |
| Pentetic Acid (Aldrich, non-USP | 0 | 0.008366 |
| Grade) | | |
| Ca(OH)₂ (Aldrich, non-USP Grade) | 0 | 0.003183 |
| NaCl (Fisher, ACS Grade) | 0.83 | 0.83 |
| Boric Acid (Mallinckrodt, ACS grade) | 0.91 | 0.91 |
| Sodium Borate | 0.11 | 0.11 |
| (Mallinckrodt, ACS Grade) | | |
| Ketotifen Fumarate (Aldrich) | 0.0043 | 0.0043 |
| Use Test (% KF Assay) | 3% | 98% |

### Example 2

Example 1 was repeated, except that ketotifen fumarate from Sifavator was used. The solutions were clear, odorless, water white and free of particulates. The ophthalmic solution was analyzed and found to have the following properties: pH of 7.2, conductivity of about 15 uS/cm, about 43 ppm ketotifen fumarate, about 104 ppm Ca₂DTPA, less than about 0.03% impurities.

We claim:

1. A method comprising mixing at least one hydroxide and pentetic acid with an ophthalmically compatible carrier and at least one oxidatively unstable component to form an ophthalmic composition comprising a pH between about 6 and about 8 and at least one salt of diethylenetriamine pentaacetic acid.

2. The method of claim 1 wherein said pH is between about 6.5 about 7.5.

3. The method of claim 1 wherein said at least one hydroxide is selected from the group consisting of calcium hydroxide, zinc hydroxide, sodium hydroxide magnesium and mixtures thereof.

4. The method of claim 1 wherein said at least one hydroxide comprises calcium hydroxide.

5. The method of claim 1 wherein said at least one salt of diethylenetriamine pentaacetic acid is present in a concentration between about 50 and about 1500 ppm.

6. The method of claim 1 wherein said at least one salt of diethylenetriamine pentaacetic acid is present in a concentration between about 100 to about 1000 ppm.

7. The method of claim 1 wherein said at least one salt of diethylenetriamine pentaacetic acid is present in a concentration between about 50 and about 500 ppm.

8. The method of claim 1 further comprising mixing into said ophthalmic composition at least one additional component selected from the group consisting of tonicity adjusting agents, buffering agents, active agents, lubricating agents, disinfecting agents, surfactants, preservatives and mixtures thereof.

9. The method of claim 5 wherein said ophthalmic composition further comprises a buffering agent selected from the group consisting of borate buffers, phosphate buffers, sulfate buffers, and mixtures thereof.

10. The method of claim 6 wherein said buffering agent comprises borate buffer or phosphate buffer.

11. The method of claim 1 wherein the oxidatively component is selected from the group consisting of acrivastine, antazoline, astemizole, azatadine, azelastine, buclizine, bupivacaine, cetirizine, clemastine, cyclizine, cyproheptadine, ebastine, emedastine, eucatropine, fexofenadine, homatropine, hydroxyzine, ketotifen, levocabastine, levoceterizine, lomefloxacin, meclizine, mepivacaine, mequitazine, methdilazine, methapyrilene, mianserin, norastemizole, norebastine, ofloxacin, oxymetazoline, pheniramine, physostigmine, picumast, promethazine, scopolamine, terfenadine, tetrahydrozoline, thiethylperazine, timolol, trimeprazine, triprolidine, pharmaceutically acceptable salts and mixtures thereof.

12. The method of claim 1 wherein the oxidatively unstable component is selected from the group consisting of acrivatine, antazoline, astemizole, azatadine, azelastine, clemastine, cyproheptadine, ebastine, emedastine, eucatropine, fexofenadine, homatropine, hydroxyzine, ketotifen, levocabastine, levoceterizine, meclizine, mequitazine, methdialazine, methapyrilene, norastemizole, norebastine, oxymetazoline, physootigmine, picumast, promethazine, scopolamine, terfenadine, tetrahyerozoline, fimilol, trimeprazine, triprolidine, and pharmaceutically acceptable salts thereof.

13. The method of claim 1 wherein the oxidatively unstable component is selected from the group consisting of phenarimine, ketotifen, ketotifen fumarate, nor ketotifen fumarate olopatadine and mixtures thereof.

14. The method of claim 1 wherein the oxidatively unstable pharmaceutical ingredient is selected from the group consisting of ketotifen, its pharmaceutically acceptable salts, and mixtures thereof.

15. The method of claim 1 wherein the oxidatively unstable component is selected from the group consisting of vitamins A, D, E, lutein, zeaxanthin, lipoic acid, flavonoids, ophthalmically compatible fatty acids, such as omega 3 and omega 6 fatty acids and combinations thereof.

16. The method of claim 1 wherein the oxidatively unstable component is selected from the group consisting of hydrogen peroxide, chlorites and mixtures thereof.
